(19) United States
(12) Patent Application Publication
(10) Pub. No.: US 2025/0345618 A1
(43) Pub. Date: Nov. 13, 2025

(54) DEFIBRILLATOR ELECTRODE PAD AND RELAY SELF-TESTS

(71) Applicant: Avive Solutions, Inc., Brisbane, CA (US)

(72) Inventors: Jimy PESIN, San Mateo, CA (US); Gregory M. FENEIS, Redwood City, CA (US); Scott Alan ELLERTHORPE, San Francisco, CA (US)

(21) Appl. No.: 19/175,858

(22) Filed: Apr. 10, 2025

Related U.S. Application Data
(60) Provisional application No. 63/646,064, filed on May 13, 2024.

(51) Int. Cl. A61N 1/39; A61N 1/02; A61N 1/04

(52) U.S. Cl. CPC A61N 1/3937; A61N 1/025; A61N 1/046; A61N 1/0492; A61N 1/3993

(57) ABSTRACT

Methods of performing defibrillator electrode pad tests and defibrillator relay tests as part of defibrillator self-tests are described. In one aspect the electrode pad tests conducted while the electrode pads are electrically isolated are used to determine the condition of the electrode pads. In another aspect, the relay is exercised during selected self-tests.

### CROSS-REFERENCE TO RELATED APPLICATIONS

This application claims the priority of U.S. Provisional Application No. 63/646,064, filed May 13, 2024, which is incorporated herein by reference in its entirety.

### FIELD

The present disclosure relates generally to defibrillator self-tests. More particularly, methods and structures for testing relays and electrode pads used in defibrillators such as automated external defibrillators (AEDs) are described.

### BACKGROUND

The electrode pads used in automated external defibrillators (AEDs) typically have a conductive gel adhered to one side of a relatively large electrode. A removeable liner covers the gel layer when the pads are stored. If/when the defibrillation electrode pads are used, the liners are removed, and the gel sides of the pads are applied to the patient. The conductive gel performs several useful functions. Initially, the gel helps the pad stick to the patient's skin thereby helping keep the pads in place during defibrillation. Additionally, the gels are electrically conductive and act as a good conductor between the electrode and the patient's skin over the entire surface area of the pads, which improves electrical conductivity between the pad and the skin and minimized skin burns.

A drawback of the conductive gel is that it tends to dry out over time which degrades both the adhesiveness and the conductivity of the pads. Therefore, the defibrillation electrode pads must periodically be replaced. The shelf lives of different electrode pads vary, but recommended shelf lives on the order of 18 months to 5 years are typical. Although defibrillation electrode pads will typically have a designated nominal useful life, in reality, the useful life of any particular electrode pad pair will vary based on storage conditions and other factors. For example, pads stored in hot and dry conditions can be expected to dry out much quicker than pads stored in cool and more humid conditions. Therefore, some defibrillators are designed to periodically “test” their pads to verify that they are still in good operating condition. To facilitate testing, the pads (i.e., a pair of pads) are stored with their conductive gels in physical contact with one another to provide electrical conductivity between the electrodes. This is typically accomplished by cutting one or more small holes in the liners so the conductive gels flow into contact through the openings. The condition of the pads can then be tested by checking the impedance of a circuit that passes through the pads. As the pads dry out over time, their impedance will increase. Thus, the condition of the pads can be monitored effectively by periodically checking the impedance of this circuit and correlating the detected impedance with a known condition of pads having that impedance. Although such testing can work well, a drawback of cutting the holes in the liners to facilitate electrical connection of the pads is that such holes tend to cause the pads to dry out more quickly than they would without the liner holes. Thus, there are continuing efforts to provide improved protocols for checking the condition of stored defibrillation electrode pads.

Most external defibrillators include a relay that is used to electrically connect the defibrillation electrode pads to a high voltage shock discharge circuit. Additionally, a patient impedance circuit may be connected to the defibrillation electrode pads via the same relay. Thus, mechanisms and protocols for checking the condition of the relay are desirable.

### SUMMARY

In one aspect, methods of determining the condition of a stored, electrically isolated pair of unopened defibrillation electrode pads installed on an external defibrillator such as an automated external defibrillator are described. An impedance measurement is made while the defibrillator is in a standby mode with the unopened electrode pads attached to the defibrillator. This measurement is taken through the electrically isolated electrode pads. The condition of the electrode pads is determined, at least in part, based on the measured impedance.

In some embodiments, the electrode pads each include an electrode, a gel layer on the electrode, and a liner over the gel layer such that the gel layer is sandwiched between the electrode and the liner. The electrode pads are stored in a storage position immediately adjacent to one another with their respective liners positioned back-to-back. In the storage position, the electrodes of the defibrillation electrode pads are electrically isolated from one another via the liners without any direct connection between their respective gel layers.

In some embodiments, the defibrillator automatically conducts impedance measurements over a period of multiple months and the condition of the defibrillation electrode pads is determined based at least in part on a multiplicity of the impedance measurements.

In some embodiments, when a determination is made that the electrode pads should be replaced based on the impedance measurement(s), a message is sent to an administrator associated with the defibrillator indicating that the defibrillator's electrode pads should be replaced.

In another aspect, methods of self-testing a defibrillator are described. The defibrillator includes a high voltage circuit, a relay, an impedance detector and a pair of defibrillation electrode pads. The high voltage circuit is electrically connected to the defibrillation electrode pads through the relay when the relay is in a first state and the impedance detector is connected to the defibrillation electrode pads through the relay when the relay is in a second state. As part of a first self-test conducted while the defibrillator is in a standby mode, a first impedance measurement is made with the relay in the first state and a second impedance measurement is made with the relay in the second state. Both impedance measurements are recorded. An electrical circuit that is made as part of the first impedance measurement passes through the electrode pads.

In some embodiments the electrodes of the defibrillation electrode pads are electrically isolated from one another via at least one insulating layer.

In some embodiments, each defibrillation electrode pad includes an electrode, a gel layer on the electrode, and a liner over the gel layer such that the gel layer is sandwiched between the electrode and the liner. During the self-test, the defibrillation electrode pads are positioned immediately adjacent one another with their respective liners positioned back-to-back and the electrodes of the defibrillation electrode pads are electrically isolated from one another via the liners without any direct connection between their respective gel layers.

In some embodiments the impedance measurements are used to determine the condition of the electrode pads. In some embodiments, the impedance measurements are used to determine the condition of the relay.

In another aspect, methods of maintaining a relay in a defibrillator are described. The defibrillator has a high voltage circuit that includes a capacitor unit capable of delivering a defibrillation shock, the relay, and a pair of defibrillation electrode pads. The high voltage circuit is electrically isolated from the defibrillation electrode pads when the relay is in a first state, and is electrically connected to the defibrillation electrode pads through the relay when the relay is in a second state. As part of a first self-test conducted while the defibrillator is in a standby mode, with the capacitor unit being uncharged, and with the defibrillation electrode pads positioned in a storage location, the relay switch is switched from the first state to the second state and thereafter back from the second state to the first state.

In some embodiments, the defibrillator is configured to periodically execute self-tests including first and second self-tests which are performed at different times, as for example, on different days. The shock delivery capacitor is not charged during any of the first self-tests and the shock delivery capacitor is at least partially charged and fully discharged during each second self-test. The relay is always maintained in the first state throughout the entirety of the second self-tests.

In some embodiments, the defibrillator conducts daily self-tests. The first self-test is executed a plurality of times each week; the second self-test is performed at most once a week; and the first and second self-tests are not conducted as part of the same daily self-test.

### BRIEF DESCRIPTION OF THE DRAWINGS

The invention and the advantages thereof, may best be understood by reference to the following description taken in conjunction with the accompanying drawings in which:

FIG. 1 is a diagrammatic representation of a modular defibrillator system architecture in accordance with one embodiment.

FIG. 2 is a block diagram illustrating electrical components of a representative defibrillator unit.

FIG. 3 is a block diagram illustrating electrical components of a representative interface unit.

FIG. 4 is a block diagram highlighting more details of the relay and impedance detection components of the defibrillator of FIG. 2.

FIG. 5 is a flow chart illustrating a method of checking impedance through a relay in accordance with one embodiment.

FIG. 6A is a diagrammatic top view of a pair of defibrillation electrode pads laid flat.

FIG. 6B is a perspective view of the pair of electrode pads of FIG. 6A folded over in the storage position.

FIG. 6C is a diagrammatic cross-sectional view of a pair of electrode pads positioned back-to-back.

In the drawings, like reference numerals are sometimes used to designate like structural elements. It should also be appreciated that the depictions in the figures are diagrammatic and not to scale.

### DETAILED DESCRIPTION

The present invention relates generally to methods and structures for testing the condition of electrode pads and relays used in defibrillators such as automated external defibrillators (AEDs).

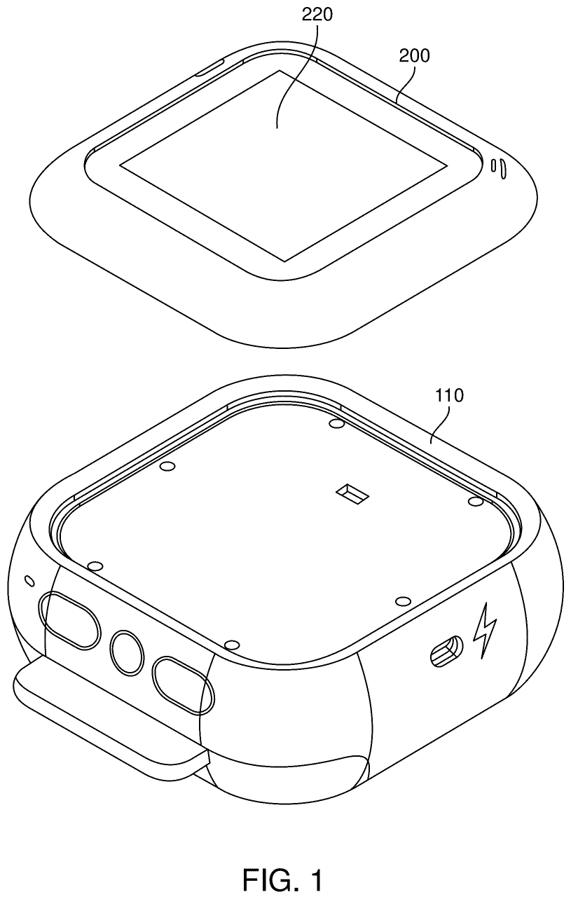

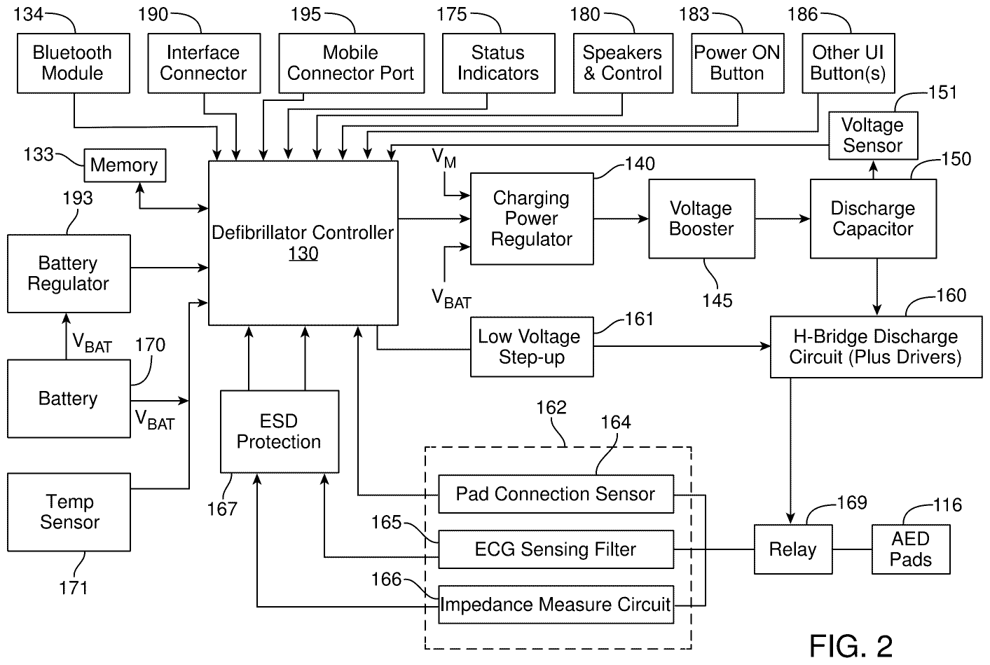

Referring initially to FIGS. 1 and 2, a defibrillator system 100 in accordance with one embodiment will be described. The illustrated defibrillator utilizes a modular architecture that is well suited for use in automated external defibrillators (including both semi-automated and fully automated defibrillators) although it may also be used in manual defibrillators and hybrid defibrillators that may be used in either automated or manual modes.

The core of the modular defibrillator system 100 is a base defibrillation unit (base unit) 110 as seen in FIG. 1. The illustrated base defibrillation unit 110 is a fully functional AED that is configured such that its functionality can be supplemented by attaching an interface unit 200 to the base unit. The base unit 110 independently functions as an AED both with and without the attached interface unit 200. In this embodiment, interface unit 200 includes one or more processors, a touch sensitive display screen 220, a communications unit and preferably its own power storage unit (e.g., battery). The touch sensitive display facilitates user interactions with the interface unit and, as appropriate, indirect interactions with the base unit. The communications unit facilitates communications with external systems and/or devices using a variety of different communications technologies and protocols, as for example, Wi-Fi communications, cellular communications, satellite communication and short-range wireless communications technologies (e.g., Bluetooth, Near Field Communication (NFC), etc.) By way of example, U.S. Pat. Nos. 10,773,091 (P006E), 10,737,105 (P006A), 11,452,881 (P016A), 11,077,312 (P016B) and U.S. patent application Ser. No. 17/007,838 (P019B), each of which is incorporated herein by reference, describe details of a variety of such modular defibrillator architectures.

FIG. 2 is a block diagram illustrating one representative electronics control architecture and associated components suitable for use in the base defibrillator unit 110. In the illustrated embodiment, the electronic components include a defibrillator controller 130, memory 133, a wireless communications module in the form of Bluetooth module 134, a charging power regulator 140, a voltage booster 145 (which may have multiple stages), a high voltage capacitor 150 for temporarily storing sufficient electrical energy suitable to provide a defibrillation shock, discharge control circuitry 1160, pad related sensing circuitry 162 and relays 169, power storage unit 170, battery regulator 193, status indicator(s) 175, speaker(s) 180 and one or more electrical connectors (e.g., interface connector 190, mobile connector port 195, charger connector (not shown), etc.). The charging power regulator 140 and voltage booster 145 which cooperate to control the charging of the shock discharge capacitor 150 are sometimes referred to herein as a charging circuit.

The defibrillator controller 130 is configured to control the operation of the base defibrillator unit and to direct communications with external devices, as appropriate. In some embodiments, the defibrillator controller includes a processor arranged to execute software (some or all of which may take the form of firmware) having programmed instructions for controlling the operation of the base unit, directing interactions with a user and communications with external components. The software may be installed on the memory 133. Although the singular term memory is often used herein, it should be appreciated that the memory may be divided into multiple different parts which take any suitable form or combination of forms (e.g., various types of RAM, ROM, PROM, EEPROM, etc.) Unless the context suggests otherwise, references to “memory” herein are intended to cover all suitable forms and combinations of physical memory. Similarly, although the singular term “processor” is often used herein, it should be appreciated that any appropriate number of processors and/or processing cores can be utilized and unless the context suggests otherwise, references to “processor” herein are intended to cover processing units composed of one or more physical processors or processing cores.

The base defibrillator unit 110 may optionally be configured so that it is capable of drawing power from certain other available power sources beyond power storage unit 170 to expedite the charging of shock discharge capacitor 150. The charging power regulator 140 is configured to manage the current draws that supply the voltage booster, regardless of where that power may originate from. For example, in some embodiments, supplemental power may be supplied from a mobile device coupled to mobile connector port 195 or from a portable charger/supplemental battery pack coupled to charger connector 197.

The voltage booster 145 is arranged to boost the voltage from the operational voltage of power storage unit 170 to the desired operational voltage of the discharge capacitor 150, which in the described embodiment may be on the order of approximately 1400V-2000V (although the defibrillator may be designed to attain any desired voltage). In some embodiments, the boost is accomplished in a single stage, whereas in other embodiments, a multistage boost converter is used. A few representative boost converters are described in the incorporated U.S. Pat. No. 10,029,109. By way of example, in some embodiments, a flyback converter, as for example, a valley switching flyback converter may be used as the voltage booster 145—although it should be appreciated that in other embodiments, a wide variety of other types of voltage boosters can be used.

A voltage sensor 151 is provided to read the voltage of the capacitor 150. The voltage sensor 151 may take the form of a voltage divider or any other suitable form. This capacitor voltage reading is utilized to determine when the shock discharge capacitor 150 is charged suitably for use. The sensed voltage is provided to controller 130 which determines when the capacitor 150 is charged sufficiently to deliver a defibrillation shock. The capacitor 150 can be charged to any desired level. This can be useful because different defibrillation protocols advise different voltage and/or energy level shocks for different conditions. Furthermore, if the initial shock is not sufficient to restart a normal cardiac rhythm, some recommended treatment protocols call for the use of progressively higher energy impulses in subsequently administered shocks (up to a point).

The discharge circuitry 160 may take a wide variety of different forms. In some embodiments, the discharge circuitry 160 includes an H-bridge along with the drivers that drive the H-bridge switches. The drivers are directed by defibrillator controller 130. The H-bridge outputs a biphasic (or other multi-phasic) shock to patient electrode pads 116 through relays 169. The relays 169 are configured to switch between an ECG detection mode in which the patient electrode pads 116 are coupled to the pad related sensing circuitry 162, and a shock delivery mode in which the patient electrode pads 116 are connected to H-Bridge to facilitate delivery of a defibrillation shock to the patient. Although specific components are described, it should be appreciated that their respective functionalities may be provided by a variety of other circuits.

The pad related sensing circuitry 162 may include a variety of different functions. By way of example, this may optionally include a pad connection sensor 164, ECG sensing/filtering circuitry 165 and impedance measurement chip/block 166. The pad connection sensor is arranged to detect whether the pads are actually connected to (plugged into) the base defibrillator unit 110. The ECG sensing/filtering circuitry 165 senses electrical activity of the patient's heart when the pads are attached to a patient. The filtered signal is then passed to defibrillator controller 130 for analysis to determine whether the detected cardiac rhythm indicates a condition that is a candidate to be treated by the administration of an electrical shock (i.e., whether the rhythm is a shockable rhythm) and the nature of the recommended shock. When a shockable rhythm is detected, the controller 130 directs the user appropriately and controls the shock delivery by directing the H-bridge drivers appropriately.

In some embodiments, the power storage unit 170 takes the form of one or more batteries such as rechargeable Lithium based batteries including Lithium-ion and other Lithium based chemistries, although other power storage devices such as one or more supercapacitors, ultracapacitors, etc. and/or other battery chemistries and/or combinations thereof may be used as deemed appropriate for any particular application. The power storage unit 170 is preferably rechargeable and may be recharged via any of a variety of charging mechanisms. In some embodiments, the power storage unit 170 takes the form of a rechargeable battery. For convenience and simplicity, in much of the description below, we refer to the power storage unit 170 as a rechargeable battery. However, it should be appreciated that other types of power storage devices can readily be substituted for the battery. Also, the singular term “battery” is often used, and it should be appreciated that the battery may be a unit composed of a single battery or a plurality of individual batteries and/or may comprise one or more other power storage components and/or combinations of different power storage units.

In some embodiments, the base defibrillator unit 110 is capable of drawing power from other available power sources for the purpose of one or both of (a) expediting the charging of shock discharge capacitor 150 and (b) recharging the power storage unit 170. In some embodiments, the battery can be recharged using one or more of the externally accessible connector ports 195, a dedicated charging station, a supplemental battery pack (portable charger), an interface unit 200, etc. as will be described in more detail below. When wireless charging is supported, the base defibrillator unit may include a wireless charging module 174 configured to facilitate inductive charging of the power storage unit 170 (e.g., using an inductive charging station 294, or other devices that support inductive charging, as for example an inductively charging battery pack, a cell phone with inductive charging capabilities, etc.).

The base unit also includes a number of software or firmware control algorithms installed in memory 133 and executable on the defibrillator controller. The control algorithms have programmed instructions suitable for controlling operation of the base unit and for coordinating the described broadcasts, as well as any point-to-point communications between the base unit 110 and the interface unit 200, connected devices, and/or any other attached or connected (wirelessly or wired) devices. These control routines include (but are not limited to): communication control algorithms, heart rhythm classification algorithms suitable for identifying shockable rhythms; capacitor charge management algorithms for managing the charging of the discharge capacitor; capacitor discharge management algorithms for managing the delivery of a shock as necessary; user interface management algorithms for managing the user instructions given by the defibrillator and/or any connected user interface devices (e.g. interface unit 200, mobile communication device 105) during an emergency; battery charge control algorithms for managing the charging of power storage unit 170; testing and reporting algorithms for managing and reporting self-testing of the base unit; software update control algorithms and verification files that facilitate software updates and the verification of the same.

In many installations, an AED will be expected to be stored for long periods of time without being plugged into power and therefore relying solely on battery power. Accordingly, it is important to minimize the power drain as much as possible during this time. In some embodiments, the defibrillator controller is configured to shut down power to all electrical components (including itself) except for (a) a real time clock (not shown), (b) the Bluetooth module 134, and (c) a power controller (not shown) when the AED is in the standby (resting) mode. In some embodiments, the clock and/or the power controller are integrated into the Bluetooth module 134 or an I/O adaptor board that includes the Bluetooth module. The clock is configured to periodically send a wake-up command to the power controller which wakes the power controller up from a sleep mode when the power controller is separate from the Bluetooth module. In response to the wakeup signal, the power controller restores power to the entire system. Once power is restored, the defibrillator controller 130 performs a status check, transmits an updated status message to the interface unit 200 and instructs the Bluetooth module 134 to update a standby status message as appropriate. After the status check is performed, the defibrillator controller will again shut down power to all electrical components except the clock, the Bluetooth module 134 and the power controller. The wakeup signals can be generated at any desired intervals, as for example, once each day.

With the described power management scheme, the Bluetooth module 134 can broadcast standby status messages even when the AED is powered down.

If the Bluetooth Module 134 makes a connection while the AED is in the standby mode, it sends a message to the power controller, which in turn, powers on the system, thereby allowing the defibrillator controller or other suitable component to communicate with the connecting device. Thus, the AED can effectively be woken up via a Bluetooth connection request. Of course, the power controller is also configured to power the system when a user presses the AED's “ON” button or otherwise activates the AED.

In other embodiments, one or more of the electrical components of the AED, such as the defibrillator controller 130, can be placed in a “sleep” mode rather than being turned off when the AED is in the standby mode. However, a significant advantage of actually turning the defibrillator controller and the bulk of the AED's electrical components off as opposed to placing them in a sleep mode is that it eliminates the power draw associated with the sleep mode, thereby potentially extending the AED's shelf life. It should be appreciated that the power controller/power down approach can be also used with AEDs that don't incorporate a Bluetooth module and/or support the low energy broadcasts described herein.

In some embodiments, a temperature sensor 171 is provided within the defibrillator itself for detecting the internal temperatures of the AED (as opposed to an environmental temperature), which is then used in the temperatures used to trigger the temperature fault notification and clearance messages. Preferrable the temperature sensor is positioned adjacent one of the more temperature sensitive components such as battery 170 so that the reported temperature is directly related to the internal temperature of the AED near the temperature sensitive component.

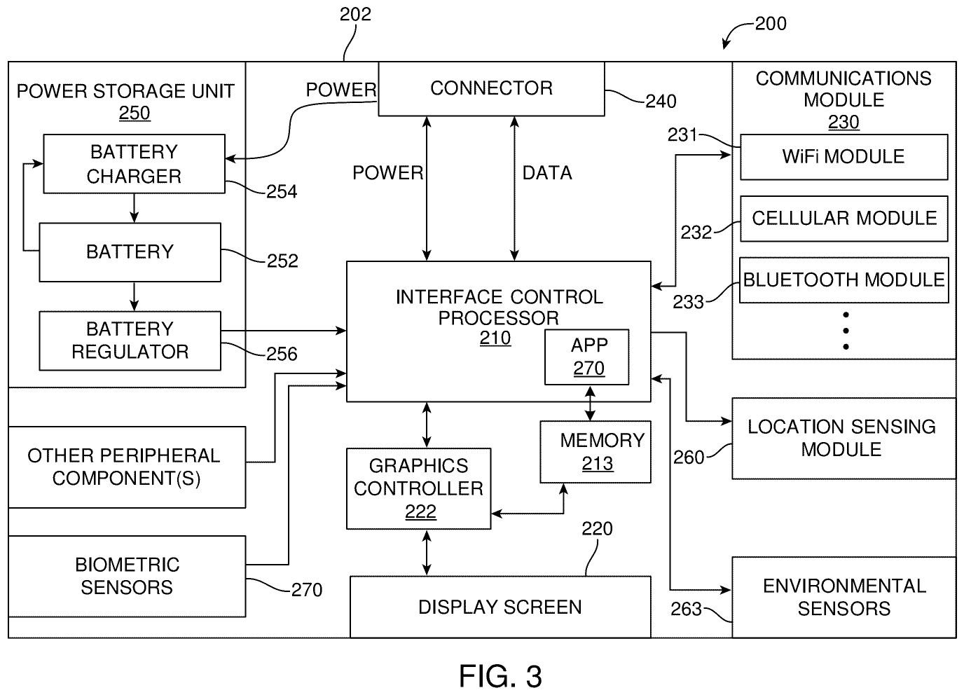

FIG. 3 illustrates some of the electrical components of a representative interface unit 200. In the illustrated embodiment, the interface unit 200 includes an interface controller (processor) 210, memory 213, a display screen 220, a communications module 230, an electrical connector 240, an interface unit power storage unit 250, and a location sensing module 260, all of which may be housed within the interface unit housing 202. The interface unit may also have software or firmware (such as an app 270) installed or installable in memory 213 having programmed instructions suitable for controlling operation of the interface unit and for coordinating communications between the interface unit 200 and the base defibrillation unit 110 and/or remote devices.

The processor 210 controls operation of the interface unit and coordinates communications with both the base unit 110 and remote devices such as a central server (as will be described in more detail below). In some embodiments, the processor 210 is arranged to execute a defibrillator app 270 or other software that can be used both during use of the defibrillator system 100 during a cardiac arrest incident and to facilitate non-emergency monitoring or/or use of the defibrillator system 100. Similar to the base unit processor 130 discussed above, unless the context suggests otherwise, the processor 210 may take the form of a single processor, multiple processors, multiple processing cores and other processing unit configurations.

The display screen 220 is a touch sensitive screen suitable for displaying text, graphics and/or video under the direction of the processor 210 to assist both during both emergency situations and at other times. The touch sensitive screen is configured to receive inputs based on a graphical user interface displayed thereon. In some embodiments an optional graphics controller 222 may be provided to facilitate communications between the interface control processor 210 and the display screen 220. In other embodiments, functionalities of the graphics controller may be part of the processor 210.

The communication module 230 is provided to facilitate communications with remotely located devices such as the central server. The communications module 230 may be configured to utilize any suitable communications technology or combination of communication technologies including one or more of cellular communications, Wi-Fi, satellite communications, Bluetooth, NFC (Near Field Communications), Zigbee communications, D (Dedicated Short-Range Communications) or any other now existing or later developed communications channels using any suitable communication protocol. By way of example, in the illustrated embodiment, the communications module 230 includes Wi-Fi, cellular and Bluetooth modules 231, 232 and 232 that facilitate Wi-Fi, cellular and Bluetooth communications respectively.

The electrical connector 240 is configured to mate with interface connector 190 on the base defibrillator unit 110. The connectors 190 and 240 are configured to facilitate communications between the defibrillator controller 130 and the interface unit's processor 210. The connectors 190 and 240 are also preferably arranged to supply power from the interface unit 200 to the base unit 110 as will be described in more detail below. In some embodiments, power will only be provided in one direction—i.e., from the interface unit 200 to the base unit 110 and not in the reverse direction during operation. A good reason for this approach is that the defibrillator is the most important component from a safety standpoint, and it is often undesirable to draw power from the base unit to power other devices (including the interface unit 200) in a manner that could reduce the energy available to charge the discharge capacitor in the event of an emergency. However, in some embodiments, the power supply may be bi-directional (at least in some circumstance) if desired—as for example if the base unit is not in use, is fully charged and plugged into an external charging power supply, etc.; or if the power passed to the interface unit is not coming from the base unit's internal battery (e.g., it is coming from a charger, a mobile communication device, or other device connected or attached to the base unit), etc..

The connectors 190 and 240 can take a variety of forms. They can be connectors with accompanying transceivers configured to handle processor level communications (such as UART, SPI, or I2C transceivers), with additional pins for power delivery (Power+GND), and connection verification (i.e. a pin that detects when there is a connection between the interface unit and the Base AED and triggers an interrupt on the Base AED signifying that there is not a unit connected). They can also be more standardized connectors such as USB connectors.

The interface power storage unit 250 provides power to operate the interface unit 210. In many embodiments, the power storage unit takes the form of a battery 252 with associated control components, although again a variety of other power storage technologies such as supercapacitors, ultracapacitors, etc. may be used in other embodiments. The associated control components may include components such as a battery charger and maintainer 254, which may include various safety monitors, and battery regulator 256. Preferably, the power storage unit 250 is rechargeable, although that is not a requirement. In some embodiments it may be desirable to utilize replaceable batteries (rechargeable or not) so that the batteries in the power storage unit 250 can be replaced when they near the end of their useful life. In some embodiments, the power storage unit 250 may also be arranged to supply supplemental power to the base unit 110. Depending on the structure and/or state of the base unit, the supplemental power can be used to help charge the discharge capacitor 150 during use; to power or provide supplemental power for the defibrillator electronics and/or to charge the base defibrillator unit's power storage unit 1170. In other embodiments, a supplemental battery within the interface unit (not shown) may be used to provide the supplemental power for the base unit rather than the power storage unit 250.

The location sensing module may incorporate a variety of technologies including Global Navigation Satellite Systems (GNSS) (e.g., GPS), Wi-Fi positioning, cellular triangulation, assisted GPS, Bluetooth Beacons, Near-Field Communications (NFC) and/or other location determining technologies. When requested, the interface unit can report its current location based on the location sensing technology that is believed to have the best accuracy under the then-present circumstances.

The interface unit may also optionally include various environmental sensors 263 and other peripheral components 266. When desired, the interface unit may include any of a wide variety of different types of sensors and peripheral components. For example, in selected embodiments, the interface unit may include one or more accelerometers and/or gyroscopes, a temperature sensor, a humidity sensor, a time of day or any other desired sensors or components.

The interface unit 200 is preferably configured to securely mechanically attach to the base unit 110. Typically, the interface unit is detachable such that it may be separated from the base unit if desired-although in other embodiments, the attachment may be more permanent in nature. The specific mechanical attachment utilized may vary widely in accordance with the needs of any particular embodiment. In some embodiments, press or form fitting attachment structures are used, while in others, latch and catch mechanisms, snap fit structures, etc. are utilized alone or in combination to releasably attach the interface unit to the base. However, it should be appreciated that a wide variety of other structures can be used in other embodiments. In some embodiments, the interface unit includes an attachment sensor (not shown) that senses when the interface unit is attached to a base unit.

In some embodiments, the interface unit may also include one or more biometric sensors 270. The biometric sensors may vary based on the needs of any particular defibrillator. Some of the biometric sensors may be suitable for use in detecting or evaluating CPR performed during emergency use of the defibrillator. Other biometrics may be useful in more general health management applications. For example, in some embodiments, the biometric sensors may include one or more of a pulse or heart rhythm sensor, a blood pressure sensor, a glucose monitor, a pulse oximeter, an ECG monitor, a sleep tracker, a thermometers, etc.

A benefit of the described modular defibrillator architecture is that the interface unit can be (and preferably is) designed to provide robust connectivity, effectively making the defibrillator a highly connected device. The relatively large touch sensitive display screen provides an interface that can be easily used by users of most any age, and the dedicated interface unit processor(s) and corresponding memory allow the interface unit to be programmed to provide a number of functionalities that are not available in defibrillators that are commercially available today.

Given that the interface unit is a connected device that has a powerful processor (or processors) and a number of familiar I/O components including, for example, a touch sensitive display screen, Wi-Fi, cellular and other wireless communications capabilities, a speaker, a microphone and optionally a camera, the interface unit can be programmed to provide a number of useful functionalities without impacting the functionality of the base defibrillator unit in any way.

In various embodiments, the interface unit processor is configured to periodically send status messages to a remotely located management server system. The management server then records the status information and can take any desired actions based thereon. In various embodiments, the management server system may take the form of a server infrastructure having one or more physical and/or virtual servers.

### Relay Testing and Impedance Detection Circuits

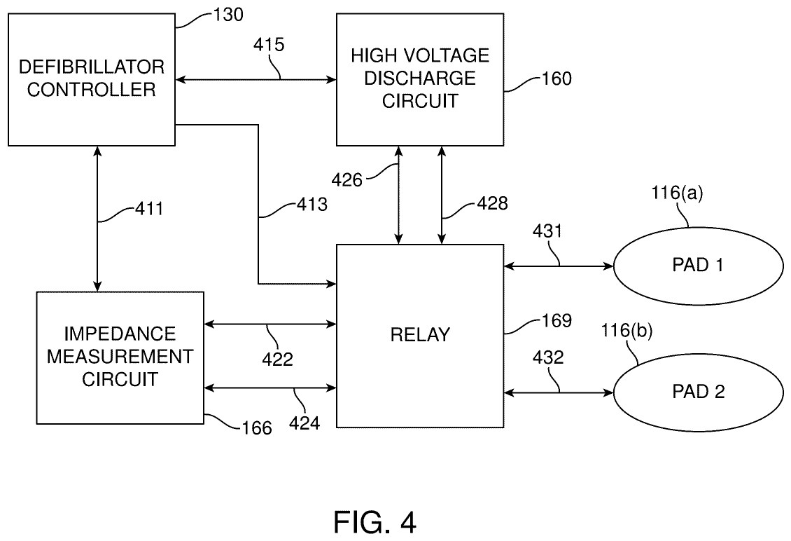

FIG. 4 is a circuit diagram showing representative relay and impedance detection components in more detail. The illustrated components include defibrillator controller 130, impedance measurement circuit 166, relay 169, first and second defibrillation electrode pads 116(a) and 116(b) and high voltage discharge circuit 160. The defibrillator controller 130 communicates with the impedance measurement circuit 166, the relay 169 and the discharge circuit 160 over lines 411, 413 and 415 respectively. Although each of these connections are represented by single lines in the drawings, it should be appreciated that in practice multiple physical lines may be provided to communicate appropriate control and, as appropriate, data, between the respective components.

Two impedance measurement I/O lines 422, 424 connect the impedance measurement circuit 166 to the relay, and two high voltage lines 426 and 428 connect the high voltage discharge circuit 160 to the relay. Additionally, wires 431 and 432 couple the relay to electrode pads 116(a) and 116(b) respectively.

The relay is configured to switch the inputs to the pads back and forth between the impedance measurement circuit 166 in a first (default) state and the high voltage discharge circuit 160 in a second state under the control of defibrillator controller 130. More specifically, in the first relay state, impedance measurement line 422 is connected to Pad 1 (116(a)) via relay 169 and wire 431, and impedance measurement line 424 is connected to Pad 2 (116(b)) via relay 169 and wire 432. In the second relay state, high voltage line 426 is connected to Pad 1 (116(a)) via relay 169 and wire 431, and high voltage line 428 is connected to Pad 2 (116(b)) via relay 169 and wire 432. Of course, in other embodiments, the relay may have additional states, as for example a state in which the pads are connected to a third input or no input.

In the illustrated embodiment, the impedance measurement circuit 166 takes the form of an integrated circuit chip configured to detect impedances. There are a number of commercially available chips that perform this function. In some circumstances, the impedance measurement functionality is the primary functionality of the integrated circuit. In other circumstances, the impedance measurement functionality may be one of several capabilities of the integrated circuit, or a minor functionality of a device having much more capability. In still other embodiments, the impedance measurement circuit may be built into defibrillator controller 130 or another component of the defibrillator.

To measure impedance, the impedance measurement generates a test signal that is output on a first one of the impedance measurement lines (e.g., line 422) which transmits the test signal to relay 169. An attenuated version of the signal is received back from the relay on the other impedance measurement line (e.g., line 424). What impedance is actually being measured will depend on the state of the relay. When the relay is in the first/default state the relay connects the pads to the impedance measurement circuit, which means that the measured impedance includes the impedance imparted by the pads. When the relay is in the second state, the measured impedance is more reflective of impedances imparted by internals of the relay and possibly the high voltage discharge circuit.

The test signal may take a variety of waveforms. By way of example, square, sinusoidal, triangular waveforms at frequencies on the order of 20-100 kHz, and preferably between 30 and 70 kHz are believed to work well. In one specific implementation, a 32 kHz square wave has been found to work well. The amplitude of the test signal can also vary. By way of example, amplitudes of 1 mA or less, or preferably in the 10-200 μA are believed to work well. In one specific implementation, an amplitude of approximately 100 μA is used.

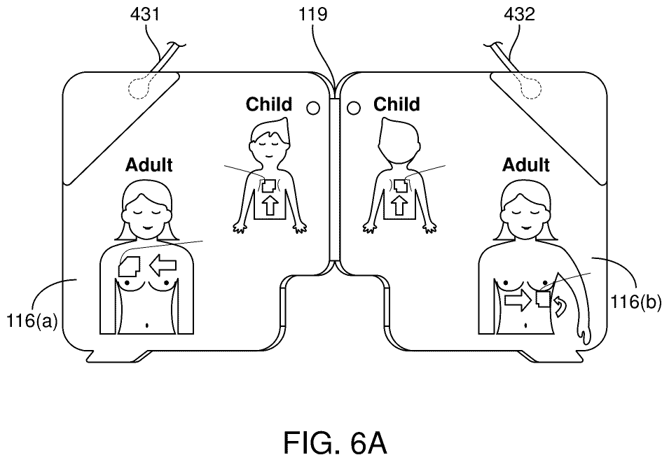

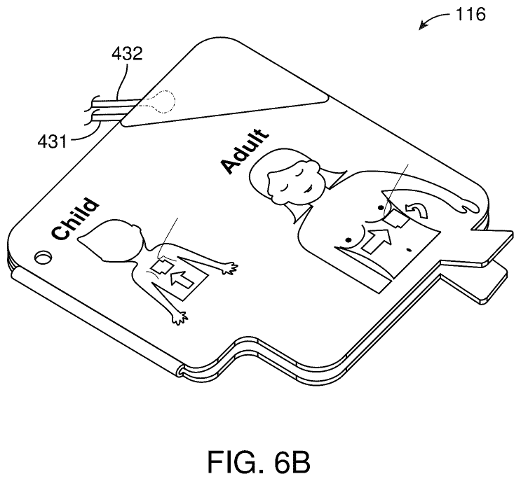

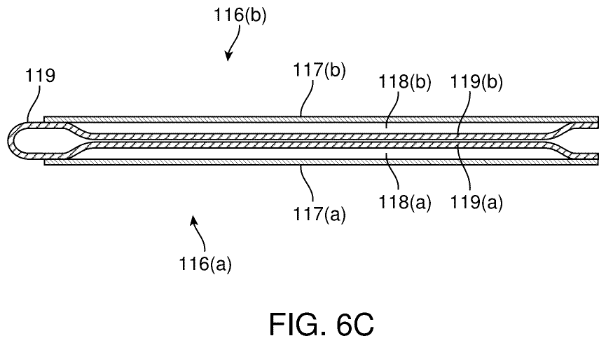

When the relay 169 connects the impedance measurement circuit 166 to the electrode pads 116, the test signal travels along a path that includes line 422, through relay 169, wire 431, pad 116(a), pad 116(b), wire 432, relay 169 and line 424 before it returns to the impedance measurement circuit. When the pads are attached to a patient, the test signal passes through the patient as it travels from Pad 1 to Pad 2. That is, the patient effectively completes the impedance detection circuit. This is how the patient impedance is determined as will be well understood by those familiar with the art. We have found that when the pads are placed adjacent one another with their respective insulative liners back-to-back (as will be described in more detail below with reference to FIG. 6) and appropriate test signals are used, the test signal readily passes from pad-to-pad to effectively complete the test circuit. This is due to the relatively high frequency of the test signal and the large surface area of the electrodes pads and occurs despite the fact that the electrode pads 116(a)&(b) are not electrically connected to one another in the classical sense.

Since a test circuit is completed, the impedance through the pads inclusive test circuit can be readily calculated. As will be described in more detail below, this type of impedance test can provide a variety of information, including, inter alia, information about: (a) the quality of the contact that the relay makes between the impedance measurement circuit 166 and the electrode pads 116; and (b) the condition of the electrode pads 116.

With respect to the quality of the contact that the relay makes between impedance measurement circuit 166, if corrosion or contamination impairs the conductivity of the relay switch, that impairment will be reflected in the measured impedance. Thus, these types of impedance measurements can be utilized to infer the condition of the relay contacts.

With respect to the condition of the electrode pads, it should be appreciated that with the pads positioned with their liners back-to-back, the “circuit” for which the impedance is measured includes the gel layer and the insulative liner of both pads. Therefore, changes in the impedance of the gel layer as it ages will impact the measured impedance of the test circuit. Typically, the impedance of the insulative liner doesn't change materially over the lifespan of the electrode pads, so that doesn't adversely affect the measurements.

Our experiments have found that for a particular pad design (e.g., the pad construction illustrated in FIG. 6), the measured impedance between different new pad pairs tends to be fairly consistent—e.g., consistent within a factor of 10% when the pads are position in an appropriate/consistent manner. This is good enough to allow the condition of both the relay and the electrode pads to be determined based on impedance measurements as will be described in more detail below.

If/when desired, the impedance can also be measured with the relay in the second state that connects the high voltage discharge circuit to the pads. This is because the frequency of the test signal is high enough that the signal will pass through some of the elements of the relay and possibly connected components sufficiently to reliably return a measurable signal to the impedance measurement circuit. This second “open circuit” measurement provides a good reference to which the first measurement can be compared.

### Relay and Electrode Pad Self-Tests

As mentioned above, an issue that can sometimes arise in defibrillator relates to corrosion or contamination of relay contacts. In one example, detected impedance is often used by AEDs to determine whether the defibrillation electrode pads have been placed on a patient. The patient impedance will vary from patient to patient depending on factors such patient size, pad placement, the types of pads used, skin conditions, etc., but impedances on the order of 25 to 200 ohms are typical. With the expected impedance being on that order, excess impedance imparted by undesired corrosion, contamination or defects in relay contacts could potentially interfere with the patient detection.

A characteristic of relays is that they tend to be self-cleaning in that in the rare circumstances that any corrosion and/or contamination were to occur on the contacts, such corrosion is typically eliminated by simply exercising the relay. That is, simply turning the relay on and off a few times tends to eliminate any contamination or corrosion from the contacts.

As will be appreciated by those familiar with the defibrillator arts, most AEDs periodically execute self-tests to ensure that the defibrillator is in good working order. The specific tests performed as part of a self-test protocol vary from manufacturer/model to manufacturer/model and the frequency at which tests performed may vary as well. In some cases, a defibrillator may perform different self-tests at different frequencies. For example, some self-tests may be performed frequently (e.g., once a day), while others are performed less frequently (e.g., once a week, once a month or on some other suitable schedule).

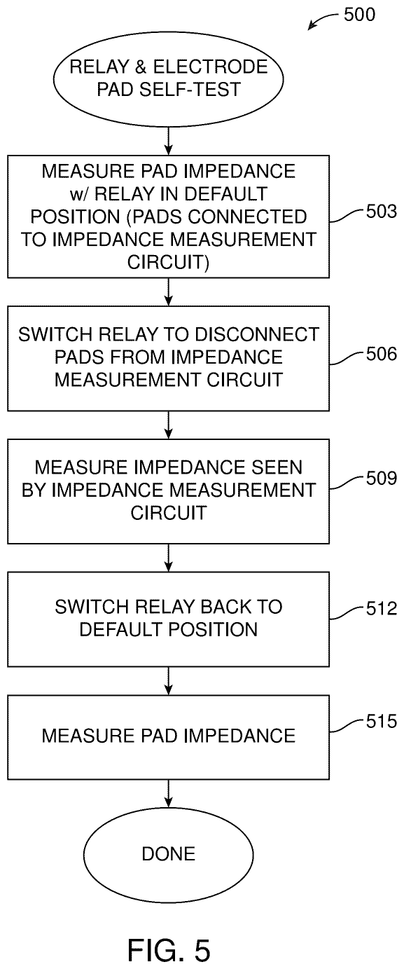

To obtain the benefits of relay self-cleaning, one or more of the defibrillator self-tests may be designed to exercise the relay. One way to exercise the relay is as a part of a pad impedance check as described below with respect to FIG. 5. However, it should be appreciated that in other embodiments, the relay can be exercised as a part of a variety of other self-test protocols. The frequency of the relay exercising self-tests can vary widely, but anything on the order of daily, weekly or monthly is believed to work well for the self-cleaning purpose.

Referring next to FIG. 5, a self-test protocol 500 suitable for both determining the condition of electrode pads 116 and testing/exercising the relay 169 will be described. Initially, in step 503, a first impedance is measured with the relay in the default position. Since the relay is in the default position, the detected impedance is for a circuit that includes the electrode pads.

By way of background, when an AED is placed at a location where it is available for emergency use, or is otherwise deployed, the electrode pads 116 are electrically connected to the AED via wires 431, 432. The pads may take a wide variety of forms physical forms. One representative pad construction is illustrated in FIGS. 6A-6C. FIG. 6A shows a pair of pads 116 positioned side-by-side on a common liner 119 having perforations laterally between the pads such that the pads can be readily separated. Each pad includes an electrode 117 having a conductive gel layer 118 thereon. A removable liner 119 extends over the gel layer 118 such that the liner cooperates with the electrode 117 to encase and preferably seal the gel layer. When the pads are stored, a fold is made along the perforations and the pads are positioned with the liners back-to-back as seen in FIGS. 6B and 6C. When an impedance test is run with the pads in this orientation, the test signal passes from the impedance measurement circuit, through the relay and wire 431 to electrode 117(a) of pad 116(a), through the conductive gel layer 118(a) and liner 119(a) of electrode pad 116(a), through the liner 119(b) and gel layer 118(b) of pad 116(b), to the electrode 117(b) of pad 116(b) and back towards the impedance measurement circuit 166 via wire 432 and the relay. Thus, the detected impedance will include the impedance of both of the gel layers and two layers of the liner.

In practice, when the relay contacts are in good order, the impedance of the gel layers and the liner(s) should be by far the largest contributors to the detected impedance. The impedance of the liner(s) should be substantially constant over time, which means that variations (increases) in the detected impedance over time are expected to directly reflect increases in the impedance of the gel layers. Gel layer impedance correlates to the condition of the gel layers since the impedance of the gel layers go up as they dry out and become less effective. Thus, periodic self-tests that incorporate the described impedance test can be used to monitor the condition of the electrode pads.

After the first impedance test has been completed, the relay is switched from the default state to a second state that disconnects the electrode pads 116 from the impedance measurement circuit 166. Block 506. Typically, this involves electrically connecting the high voltage discharge circuit 160 to the electrode pads which is fine because during the relay test, there is no charge within the high voltage discharge circuit or the discharge capacitor 150. However, if the relay supports other states, a switch could be made to a third state.

After the switch is made to the second state, another impedance measurement is taken. Block 509. Because of the high frequency of the test signal, current will find a way through the relay and possibly other components suitably to obtain an impedance measurement. As long as the relay is switching properly, the detected impedance will be consistent, so this second impedance measurement step provides a mechanism for verifying that the relay is switching properly and disconnected from the impedance measurement circuit 166.

After the second impedance measurement, the relay is switched back to the default position. Block 512. Optionally, a third impedance measurement can be taken at this point. Block 515. The third impedance measurement is expected to replicate the first impedance measurement so its results can both (a) verify the first measured pad impedance, (b) confirm that the relay is switching properly, and (c) demonstrate effective contact cleaning by measuring a decrease in impedance from the first measured impedance if the first measured impedance was similar to the second impedance measurement.

Each of the detected impedances is recorded in appropriate memory within the AED and preferably is also reported to an appropriate management node—which can be a defibrillator management server, a defibrillator management app on a smartphone or other device associated with the defibrillator, an analyzer on the defibrillator itself, or any other node that is responsible for analyzing the measured impedance to determine the condition or health of the electrode pads and/or relay based on these measurements. Block 518.

It should be apparent that performing the impedance measurement self-test protocol of FIG. 5 inherently exercises the relay, which effectively eliminates or prevents the buildup of any contaminants or corrosion on the relay contacts, which helps ensure that the relay works well throughout the operational life of the AED.

The frequency that the impedance measurements are performed may vary widely based on the needs of any particular design. By way of example, frequencies on the order of daily to monthly work well for both relay maintenance and pad condition monitoring.

Some defibrillator self-test protocols call for periodically charging and internally discharging the discharge capacitor 150 to facilitate testing the charging circuitry, the discharge capacitor and the high voltage discharge circuit. The capacitor charging tests may be done less frequently than the daily self-tests often performed by AEDs to preserve the life of the battery 170. For example, the capacitor charging tests may be performed on a monthly, weekly, or other non-daily basis. In some embodiments, the impedance measurement tests are only run as part of test suites that do not include capacitor charging test. For example, if/when the capacitor charging tests are performed on a weekly basis, the impedance measurement tests may be executed as part of daily self-tests the other six days of the week, but not on the day that the capacitor charging test occurs. This provides additional layers of safety to help ensure that a residual high voltage charge isn't inadvertently applied to the pads 116 during the relay test.

The impedance measurements made during the pad and relay test are used to determine the condition of the electrode pads 116 and/or the relays 169 as represented by block 521. These analysis may be performed by any node in the system that has access to the relevant data, including, the AED itself, a management server/server infrastructure, a mobile device running an AED management app, or any other suitable system.

The described pad and relay tests can be used for a variety of purposes. For example, pad aging can be monitored by observing trends (changes in) the impedance values detected over time in the measurements with the relay in the first state (i.e., with the pads are electrically coupled to impedance measurement circuit 166 in the first (default) state)). The pad aging process is gradual and as the gel layer begins to dry out, the impedance of the pads will gradually increase. Therefore, gradual changes (increases) in the measured impedance over time are indicative of the condition of the electrode pads.

In general, pads of the same type may be expected to age at somewhat consistent rates when all other factors are equal. However, pads stored in unfavorable environments (e.g., in hotter or dryer environments) tend to age more quickly. Thus, a trend of the detected impedance increasing at a faster rate than expected may indicate that the pads are being stored in unfavorable environments. The management node can proactively notify the administrator/owner of that occurrence and/or that pad replacement may be required sooner than normal.

In some embodiments, for a particular type of pad, a first impedance threshold may be identified that is deemed to indicate that it is time to replace the electrode pads. Thus, when the management node observes a historical trend of gradually increasing impedances over time, it may be deemed time to replace the pads when the detected impedance reaches the first threshold. At such time, the management node may send a notification to the AED's administrator/owner indicating that it is time to replace the electrode pads/pad cartridge. If the electrode pads are not timely replaced and the impedance continues to rise over time to reach a second threshold, the pads may be deemed nonfunctional and the management server may so notify the administrator/owner. Of course, any of a variety of different thresholds or trends may be used in determining the types of notifications to send, and when to send notifications to the administrator/owner.

In practice, the approved shelf life of conventional electrode pads are determined based on aging estimates that don't take the impacts of individual variations in storage conditions or manufacturing variations for different pads into account. Therefore, some pads may be perfectly adequate for use for some time beyond their designated shelf life, while others stored in more adverse conditions may degrade to the point that they should be replaced before the functionality before their designated shelf life occurs. When the condition of the electrode pads can be reliably monitored as described, this opens the possibility of extending the permitted shelf life of pads that remain in good operating condition, while affirmatively identifying pads that are aging (or otherwise impaired for any reason) to the point that they should be replaced.

The determination of the pad conditions may be made by the AED itself, the management server or any other appropriate node. When the AED is configured to regularly report the impedances detected during the relay and electrode pad self-tests, to a management server, it is sometimes advantageous to have the management server perform the analysis of the pads condition so that the AED is not required to have analytic software installed thereon that regularly performs the pad condition analysis.

In another example, acute changes to impedance from self-test to self-test would indicate failures in the relay. Therefore, impedance values detected with the relay in the first state above a predetermined relay fault threshold indicates that default contacts have failed. Impedance values below the predetermined threshold in second state indicate that the relay armature has failed, and the relay cannot change positions. Both conditions could indicate that the device is not operable. Therefore, in some embodiments, the AED is deemed to have failed a self-test if either of those conditions are met. In some embodiments, when a self-test failure occurs, a status message is sent to the management server indicating that the self-test failure occurred and appropriate indicator lights on the AED are lit in a manner that indicates that the AED has failed a self-test. In turn, the management server may send a notification to the AED's administrator indicating that there has been a self-test failure. Typically, relay failures would be detected by the AED itself. However, if a relay failure is determined by another node, an appropriate message can be sent to the AED to cause it to light the appropriate indicator light.

It should be appreciated that the specific impedance values for the pad condition thresholds will vary based on pad type and packaging, as well as design goals. Thus, the appropriate impedance thresholds for any particular type of pad/packaging may be determined experimentally. Appropriate relay fault threshold may be determined experimentally as well.

### Example #1

In one test case, a large number of unopened electrode pad pairs positioned back-to-back as shown in FIG. 6B were determined to have an average impedance measured by a defibrillator on the order of 4000 ohms when new, and a measured impedance on the order of 7000 ohms when it was deemed that they should be replaced. For a defibrillator having such electrode pads, a fault threshold of 7000 ohms may be used for impedance measurements taken with the relay in the default (first) state. If/when the defibrillator detects that the 7000 ohm fault threshold is exceeded, it fails the self-test and triggers indicator lights accordingly. From a diagnostics standpoint, if the change to 7000+ ohm occurred gradually over time as the pads aged, then the fault may be deemed to be due to expired pads. Alternatively, if there was a sudden, large change in measured impedance, the fault may be deemed to be the result of a relay fault.

In this example, a lower impedance threshold, as for example a measurement on the order of 6500 ohms may be used to mark the pads as ready for replacement soon.

For any given pad/packaging type, a profile can be created that shows statistically what the expected impedance is over time. If/when the management node determines that the pads are aging faster than expected (which may be due to the defibrillators storage in hot and/or dry conditions), notifications may be sent to the defibrillator's administrator indicating that the current rate, it can be expected that the pads will be compromised in X-months or other providing other predictive information.

Although only a few embodiments of the invention have been described in detail, it should be appreciated that the invention may be implemented in many other forms without departing from the spirit or scope of the invention. The description above focuses primarily on automated external defibrillators-however it should be appreciated that the described techniques can be used in conjunction with various other external defibrillators with unopened electrode pads as well. Therefore, the present embodiments should be considered illustrative and not restrictive, and the invention is not to be limited to the details given herein, but may be modified within the scope and equivalents of the appended claims.

What is claimed is:

1. A method of determining a condition of a stored, electrically isolated, unopened pair of defibrillation electrode pads installed on a defibrillator, the method comprising:
while the defibrillator is in a standby mode with the electrically isolated, unopened defibrillation electrode pads connected to the defibrillator, measuring an impedance through the electrically isolated, unopened defibrillation electrode pads; and
determining the condition of the electrically isolated, unopened defibrillation electrode pads based at least in part on the measured impedance.

2. A method as recited in claim 1 wherein:
each defibrillation electrode pad includes an electrode, a gel layer on the electrode, and a liner over the gel layer such that the gel layer is sandwiched between the electrode and the liner;
the defibrillation electrode pads are stored in a storage position immediately adjacent one another with their respective liners positioned back-to-back;
in the storage position, the electrodes of the defibrillation electrode pads are electrically isolated from one another via the liners without any direct connection between their respective gel layers.

3. A method as recited in claim 1 further comprising:
by the defibrillator, automatically conducting a series of the impedance measurement over a period of multiple months;
recording the associated measured impedances; and
determining the condition of the defibrillation electrode pads based at least in part on a multiplicity of the impedance measurements.

4. A method as recited in claim 3, wherein:
after at least some of the impedance measurements, the defibrillator directly or indirectly conveys the measured impedance to a management server system, and wherein the management server system records the measured impedance; and
the management server system makes the determination of the condition of the electrode pads base on least in part on a plurality of the measured impedances received from the defibrillator.

5. A method as recited in claim 4 wherein the determination of the condition of the electrode pads includes a determination of whether the defibrillation electrode pads should be replaced.

6. A method as recited in claim 1 wherein when the determined condition is that the defibrillation electrode pads should be replaced, the method further comprises:
sending a message to an administrator associated with the defibrillator indicating that the defibrillator's electrode pads should be replaced.

7. A method as recited in claim 6 wherein the message is sent to the administrator via at least one of:
a text message;
an e-mail message; and
a notification in a management platform user interface.

8. A method as recited in claim 6 wherein the message is sent to the administrator by one selected from the group consisting of:
the defibrillator;
a management server system that receives a status message that contains the measured impedance from the defibrillator; and
an app executing on a mobile device that receives the measured impedance from the defibrillator.

9. A method as recited in claim 1 wherein the impedance is measured by an impedance measurement circuit configured to detect an impedance of a patient through the defibrillation electrode pads when the defibrillator is in use.

10. A method of self-testing a defibrillator having a high voltage circuit, a relay, an impedance detector and a pair of defibrillation electrode pads, the high voltage circuit being electrically connected to the defibrillation electrode pads through the relay when the relay is in a first state and the impedance detector being connected to the defibrillation electrode pads through the relay when the relay is in a second state, the method comprising:
as part of a first self-test conducted while the defibrillator is in a standby mode, measuring a first impedance with the relay in the first state and recording the first measured impedance; and
as part of the first self-test, measuring a second impedance with the relay in the second state and recording the second measured impedance.

11. A method as recited in claim 10 further comprising conducting a series of self-tests, each self-test including the measurement and recordation of corresponding first and second impedances measurement, wherein the self-tests are conducted over multiple days.

12. A method as recited in claim 11 further comprising determining a condition of the electrode pads based at least in part on one or more of the impedance measurements.

13. A method as recited in claim 10 further comprising determining that the relay is faulty based at least in part on one or more of the impedance measurements.

14. A method as recited in claim 12 wherein:
each defibrillation electrode pad includes an electrode, a gel layer on the electrode, and a liner over the gel layer such that the gel layer is sandwiched between the electrode and the liner;
during the self-test, the defibrillation electrode pads are positioned immediately adjacent one another with their respective liners positioned back-to-back;
during the self-test, the electrodes of the defibrillation electrode pads are electrically isolated from one another via the liners without any direct connection between their respective gel layers.

15. A method of maintaining a relay in a defibrillator having a high voltage circuit that includes a capacitor unit capable of delivering a defibrillation shock, a relay, and a pair of defibrillation electrode pads, the high voltage circuit being electrically isolated from the defibrillation electrode pads when the relay is in a first state, and the high voltage circuit being electrically connected to the defibrillation electrode pads through the relay when the relay is in a second state, the method comprising:
as part of a first self-test conducted while the defibrillator is in a standby mode, the capacitor unit is uncharged, and with the defibrillation electrode pads positioned in a storage location, causing the relay to switch from the first state to the second state and thereafter causing the relay to switch back from the second state to the first state.

16. A method as recited in claim 15 wherein the defibrillator periodically conducts self-tests, including periodically executing the first self-test and periodically executing a second self-test, the first and second self-tests being performed at different times, wherein:
the shock delivery capacitor is not charged during any of the first self-tests; and
the shock delivery capacitor is at least partially charged and fully discharged during each second self-test and the relay is always maintained in the first state throughout an entirety of the second self-tests.

17. A method as recited in claim 16 wherein the defibrillator conducts daily self-tests with the first self-test being executed a plurality of times each week and the second self-test being performed at most once a week, and wherein the first and second self-tests are not conducted as part of the same daily self-test.

18. An automated external defibrillator (AED) comprising:
a high voltage circuit that includes a capacitor unit capable of delivering a defibrillation shock;
a pair of defibrillation electrode pads; and
a relay, the high voltage circuit being electrically isolated from the defibrillation electrode pads when the relay is in a first state, and the high voltage circuit being electrically connected to the defibrillation electrode pads through the relay when the relay is in a second state; and
wherein the AED is configured to execute a self-test that exercises the relay by at least one of (i) switching from the first state to the second state, and (ii) switching from the second state to the first state.

19. An automated external defibrillator (AED) comprising:
a high voltage circuit that includes a capacitor unit capable of delivering a defibrillation shock;
a pair of defibrillation electrode pads;
an impedance measurement circuit; and
a relay, wherein when the relay is in a first state, the impedance measurement circuit is electrically coupled to the defibrillation electrode pads with the high voltage circuit electrically isolated from the defibrillation electrode pads when the relay is in a first state, and the high voltage circuit being electrically connected to the defibrillation electrode pads through the relay when the relay is in a second state; and
wherein the AED is configured to execute a self-test that exercises the relay by at least one of (i) switching from the first state to the second state, and (ii) switching from the second state to the first state.

20. A method of determining a condition of a stored, electrically isolated, unopened pair of defibrillation electrode pads installed on a defibrillator, the method comprising:
while the defibrillator is in a standby mode with the electrically isolated, unopened defibrillation electrode pads connected to the defibrillator, measuring an impedance through the electrically isolated, unopened defibrillation electrode pads; and
determining the condition of the electrically isolated, unopened defibrillation electrode pads based at least in part on the measured impedance.

21. A method as recited in claim 20 wherein the condition is determined based on the measured impedance exceeding a designated impedance threshold, together with an analysis of a series of previous impedance measurements that together show that the impedance of the pads had gradually deteriorated over time.
